(19) United States
(12) Patent Application Publication
(10) Pub. No.: US 2011/0245791 A1
(43) Pub. Date: Oct. 6, 2011

(54) Diaper Shield

(76) Inventor: Melanie C. Miller, South Jordan, UT (US)

(21) Appl. No.: 13/078,732

(22) Filed: Apr. 1, 2011

Related U.S. Application Data
(60) Provisional application No. 61/320,254, filed on Apr. 1, 2010.

(51) Int. Cl. A61F 13/84

(52) U.S. Cl. 604/385.01

(57) ABSTRACT

A diaper shield includes an outer cover and a liner. The outer cover has a substantially rectangular center panel, which has a first and a second longitudinal edges, and a first and a second lateral side edges. A first substantially triangular panel is connected to the first lateral side. A second substantially triangular panel is connected to the second lateral side. A first strap is connected to the first triangular panel extending longitudinally outward. A second strap is connected to the second triangular panel extending longitudinally outward. The liner, which defines a bodyfacing surface and includes a fluid permeable material, is connected to the outer cover.

### CROSS-REFERENCE TO RELATED APPLICATIONS

This application claims the benefit of U.S. Provisional Patent Application No. 61/320,254, entitled “a diaper that covers a baby's back, preventing the mess from a ‘blowout’, leakage out the backside of a standard diaper”, filed on Apr. 1, 2010, which is incorporated herein by reference in its entirety.

### BACKGROUND OF THE INVENTION

1. The Field of the Invention

The invention relates to diaper shields, particularly diaper shields that can absorb and retain waste expelled by a wearer that is not captured by a standard diaper.

2. The Relevant Technology

Standard diapers, under certain situations, may not contain the waste produced by the wearer resulting in contamination of the garments worn by the wearer. Undesired leakage of the diaper requires replacement of the wearer's garments. Accordingly, there remains a need for a diaper shield for use in conjunction with standard diapers that improves the ability to contain the waste, and reduces waste leakage.

U.S. Patent Publication 2005/0132476 disclosed a waist protection garment. The waist protection garment may improve the fit of the standard diapers, and improve the security of fasteners on the standard diapers. U.S. Patent Publication 2008/0039813 disclosed a diaper pad adapted to absorb and retain waste expelled by an infant that is not captured by the standard diaper. The diaper pad is attached to the rear waistband of a standard diaper to provide a leak-proof barrier between the infant's back and the article of clothing that the infant is wearing.

### BRIEF SUMMARY OF THE INVENTION

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In one embodiment, a diaper shield includes an outer cover and a liner. The outer cover has a substantially rectangular center panel, which has a first and a second longitudinal edges, and a first and a second lateral side edges. A first substantially triangular panel is connected to the first lateral side. A second substantially triangular panel is connected to the second lateral side. A first strap is connected to the first triangular panel extending longitudinally outward. A second strap is connected to the second triangular panel extending longitudinally outward. The liner, which defines a bodyfacing surface and includes a fluid permeable material, is connected to the outer cover.

In another embodiment, a diaper shield includes an outer cover and a liner. The outer cover has a substantially rectangular center panel, which has a first and a second longitudinal edges, and a first and a second lateral side edges. A first substantially triangular panel is connected to the first lateral side. A second substantially triangular panel is connected to the second lateral side. A first strap is connected to the first triangular panel extending longitudinally outward. Concave shaped peripheral side edge extends between the first strap and the first triangular panel. A second strap is connected to the second triangular panel extending longitudinally outward. Concave shaped peripheral side edge extends between the second strap and the second triangular panel. The liner, which defines a bodyfacing surface and includes a fluid permeable material, is connected to the outer cover.

In one embodiment, a diaper shield includes an outer cover, a liner, and a fastener. The outer cover has a center panel, which has opposite a first side edge and a second side edge. A first strap is connected to the center panel at the first side edge. A second strap is connected to the center panel at the second side edge. The liner, which defines a bodyfacing surface and includes a fluid permeable material, is connected to the outer cover. The fastener may connect the first strap and the second strap. The diaper shield also provides means for reducing the center panel's tendency to rotate and move around when worn by a wearer.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 illustrates a perspective view of one embodiment of the present invention;

FIG. 2 illustrates a front view of the embodiment illustrated in FIG. 1;

FIG. 3 illustrates a back view of the embodiment illustrated in FIG. 1;

FIG. 4 illustrates a left side view of the embodiment illustrated in FIG. 1;

FIG. 5 illustrates a right side view of the embodiment illustrated in FIG. 1;

FIG. 6 illustrates a top view of the embodiment illustrated in FIG. 1;

FIG. 7 illustrates a bottom view of the embodiment illustrated in FIG. 1;

FIG. 8 illustrates a perspective view of a child wearing the diaper shield;

FIG. 9 illustrates an assembly view of the embodiment illustrated in FIG. 1;

FIG. 10 illustrates a plan view of another embodiment of the diaper shield; and

FIG. 11 illustrates a plan view of further embodiment of the diaper shield.

### DETAILED DESCRIPTION OF THE VARIOUS EMBODIMENTS

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

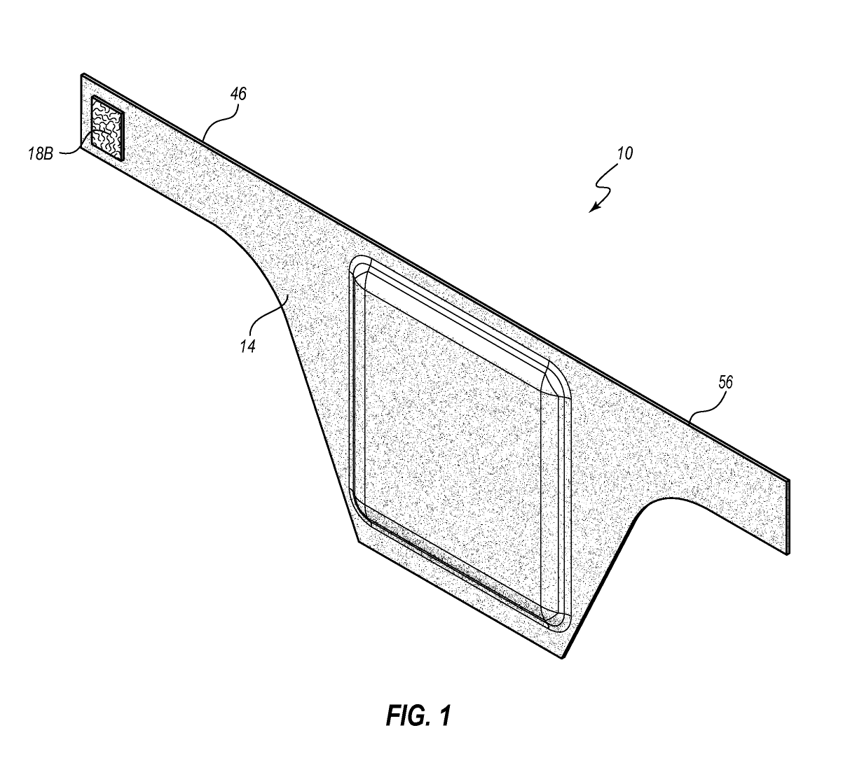

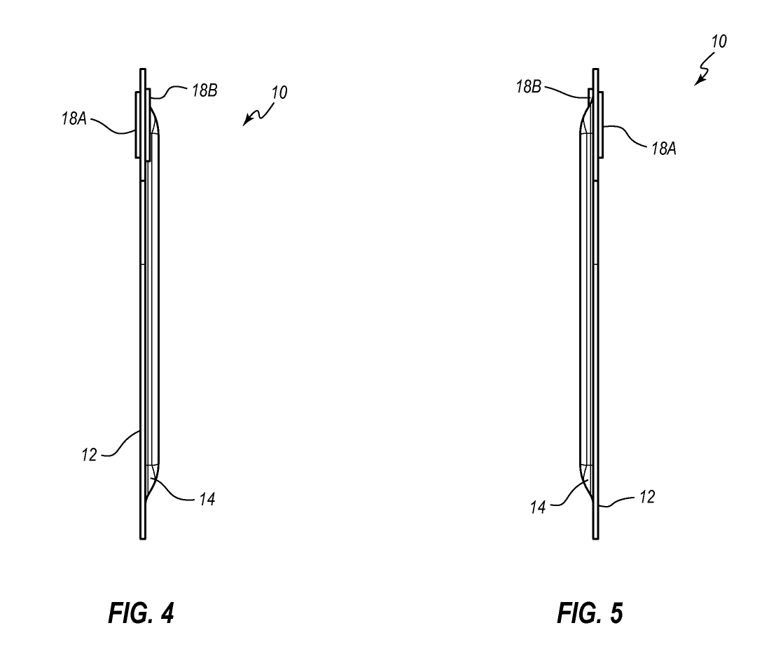

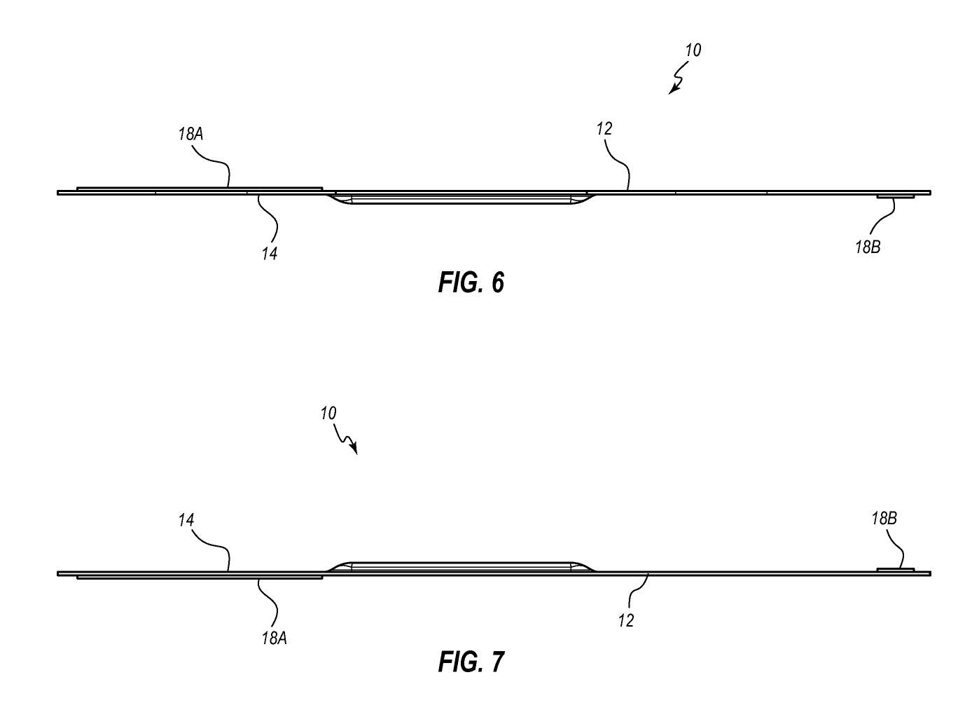

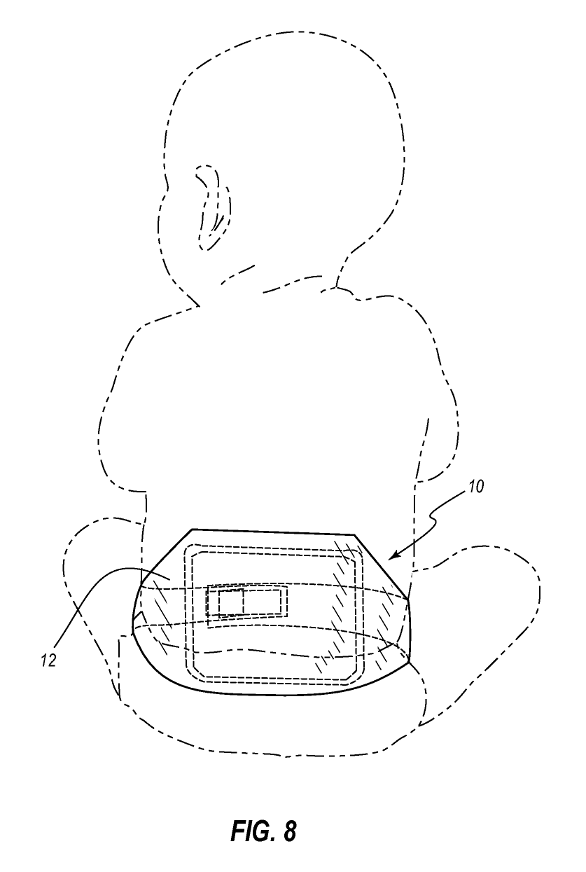

Referring to FIGS. 1-9, various embodiments of a diaper shield 10 may include an outer cover 12, a liner 14, an absorbent core 16, and a fastener 18A-B. The liner 14 may be attached to the outer cover 12 with a bonding agent or adhesive, sonic bonds, thermal bonds, pinning, stitching or any other attachment techniques known in the art, as well as combinations thereof. The liner 14 and the outer cover 12 may also be formed integrally from a single planar material. The liner 14 may be in superposed relation to the outer cover 12. The peripheral edges of the liner are attached to the outer cover along the liner's peripheral edges. The liner and the outer cover may be connected along the liner's other regions. The liner may optionally cover an area which is larger or smaller than the area of the outer cover, as desired.

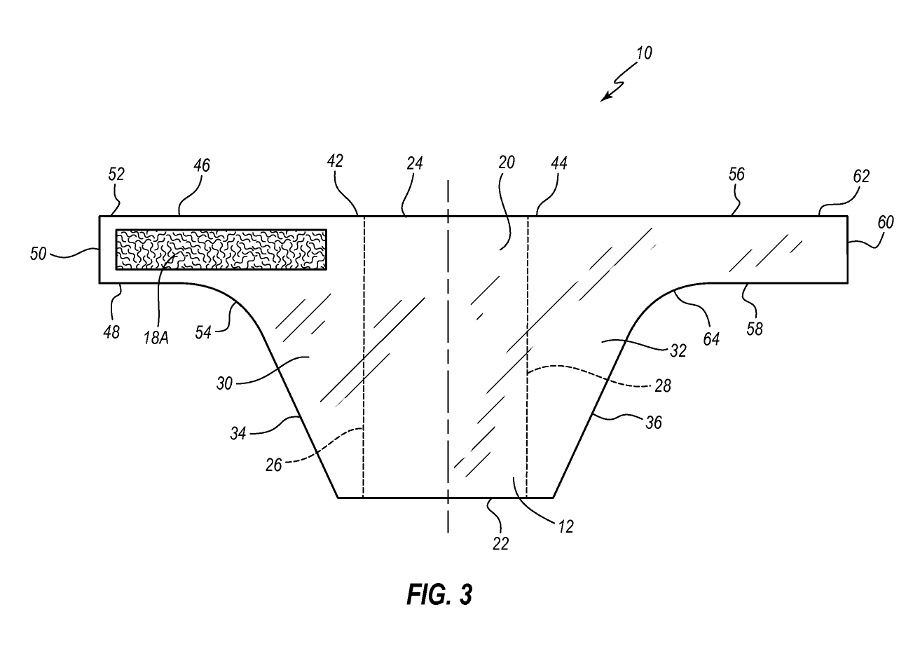

Referring to FIG. 3, the outer cover 12 has a substantially rectangular center panel 20 with a first longitudinal side edge 22, a second longitudinal side edge 24, a first lateral side edge 26, and a second lateral side edge 28. The longitudinal side edges 22, 24 of the center panel may have a stitched gathering or elastic band to prevent leakage. The center panel may be about 4-10 inches in longitudinal length, and about 4-10 inches in lateral length.

A first side panel 30 is connected to the first lateral side edge 26 of the center panel and extends longitudinally outward. A second side panel 32 is connected to the second lateral side edge 28 and extends longitudinally outward. The side panels may be configured in a generally triangular or curved shape. The center panel and side panels may also be configured to define a trapezoidal shape. First edges 34, 36 of the side panels 30, 32, respectively, may extend outwardly at about a 30-60 degree angle from the lateral sides 26, 28 to form the triangular or curved shape in the side panels 30, 32. The whole or part of the lateral sides 26, 28 may define second edges of the side panels 30, 32 respectively. Third edges 42, 44 of the side panels 30, 32 extend longitudinally outward from the lateral sides 26, 28 respectively. The third edges 42, 44 may extend out from the side panels 30, 32 approximately 6-8 inches, or otherwise, configured to be long enough to wrap around to the wearer's waist. The third edges 42, 44 may be collinear with the second longitudinal side edge 24 of the rectangular center panel 20. Optionally, the third edges 42, 44 are not collinear with the second longitudinal side edge 24. The two side panels 30, 32 may have identical or different shapes.

A first strap 46 is connected to the first side panel 30 and extends longitudinally outward from the first side panel 30. The first strap 46 has a first edge 48, a second edge 50, and a third edge 52. A concave shaped peripheral side edge 54 is configured and extends between the first edge 34 of the first side panel and the first edge 48 of the first strap to define a radius. Optionally, the first edge 34 of the first side panel and the first edge 48 of the first strap can be connected in a straight line. The third edge 52 of the first strap may be collinear with the second longitudinal side edge 24 of the center panel. Optionally, the third edge 52 of the first strap is not collinear with or is offset from the second longitudinal side edge 24 of the center panel. The second edge 50 connects the first edge 48 and the third edge 52 and extends laterally to define a rectangular projection.

A second strap 56 is connected to the second side panel 32 and extends longitudinally outward from the second side panel 32. The second strap 56 has a first edge 58, a second edge 60, and a third edge 62. A concave shaped peripheral side edge 64 is configured and extends between the first edge 36 of the second side panel and the first edge 58 of the second strap to define a radius. Optionally, the first edge 36 of the second side panel and the first edge 58 of the second strap can be connected in a straight line. The third edge 62 of the second strap may be collinear with the second longitudinal side edge 24 of the center panel. Optionally, the third edge 62 of the second strap is not collinear with or is offset from the second longitudinal side edge 24 of the center panel. The second edge 60 connects the first edge 58 and the third edge 62 and extends laterally to define a rectangular projection. The second edge 50 of the first strap and the second edge 60 of the second strap may define the longitudinal length of the diaper shield.

The shape of the first and second side panels is beneficial in that they may reduce the center panel's tendency to rotate, flip and/or move around when worn by a wearer, and allow the diaper shield to closely conform to the wearer. The second strap's length may be longer, shorter, or equal to the length of the first strap. When the first and second straps are connected, they may merely meet or overlap with each other.

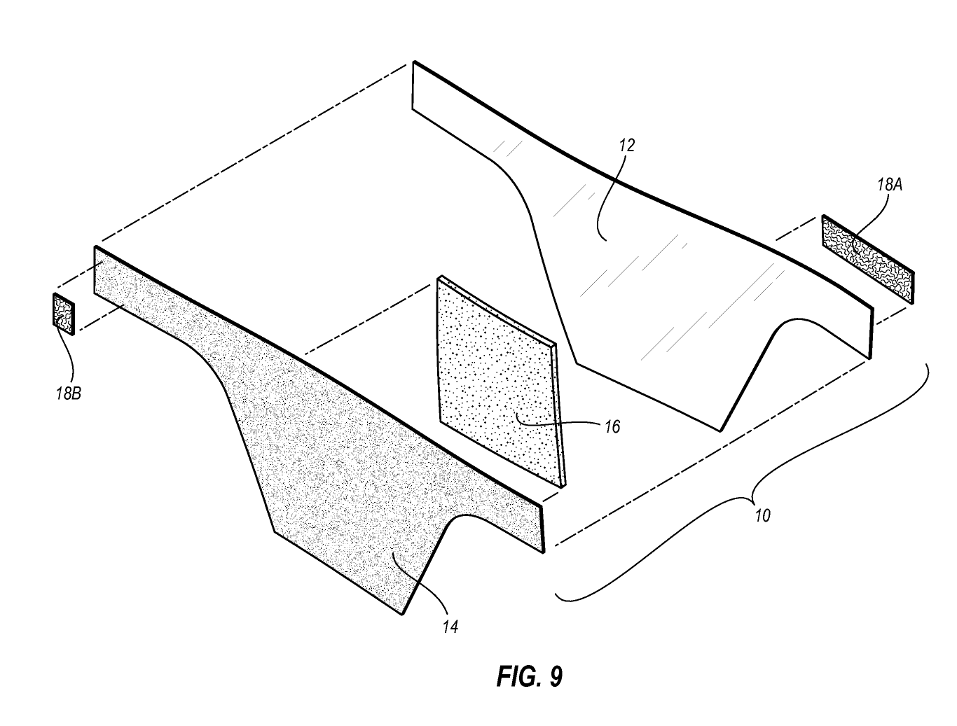

Referring to FIGS. 1 and 9, the absorbent core 16 is sandwiched between or disposed between the liner 14 and the outer cover 12. The outer cover and the liner extend outward beyond peripheral edge of the absorbent core. The absorbent core may be attached to the outer cover and the liner with a bonding agent or adhesive, sonic bonds, thermal bonds, pinning, stitching or any other attachment techniques known in the art, as well as combinations thereof. In another embodiment, the absorbent core is not attached to either the outer cover or the liner, and is merely sandwiched between the outer cover and the liner in a free floating arrangement. The absorbent core may be in a substantially rectangular, trapezoid, or curved shape.

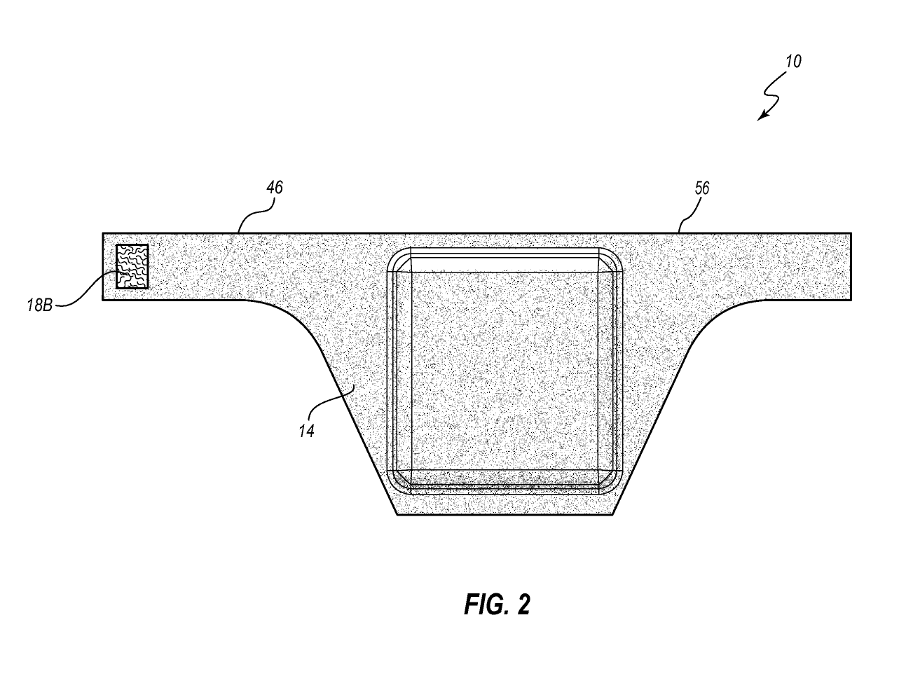

The fastener 18A-B may be releasably engageable allowing for securing and removing the diaper shield from the torso of the wearer. The diaper shield may be adjustable in such a way that a diaper shield of a given length may be used with wearers of different body sizes. In other words, the circumference of the diaper shield when worn can be adjusted to fit various waist sizes. The fastener may include any suitable fastener such as adhesive tape tab fasteners, hook and loop fasteners, mushroom fasteners, snaps, pins, and the like, and the combination thereof. In one embodiment, referring to FIGS. 2-3, a relatively long strip of fastener material 18A (e.g. hook and loop material) attached to the outer cover's first strap 46 near the first strap's second edge 50. A relative short strip 18B of cooperating fastener material (e.g. loop material) is attached to the liner's second strap near the second strap's second side edge 60. With this configuration, the fastener material 18A would engage the cooperating fastener material 18B at various positions.

In one embodiment, to allow the diaper shield to be adjustable, it may have a fastener material that extends along the side edge 42, for example, away from the second edge 50 for several inches, and may be attached on the liner side or the outer cover side of the diaper shield. For instance, the fastener material may extend for about a half an inch to six inches from the side edge 50, along the outer cover of the diaper shield. Meanwhile, a corresponding fastener material may be disposed at or near the opposite side edge 60 such that when the diaper shield is disposed about a wearer's waist, the fastener material engages the corresponding fastener material. The length of the fastener material and its exact location are dependent upon the desired adjustability of the diaper shield.

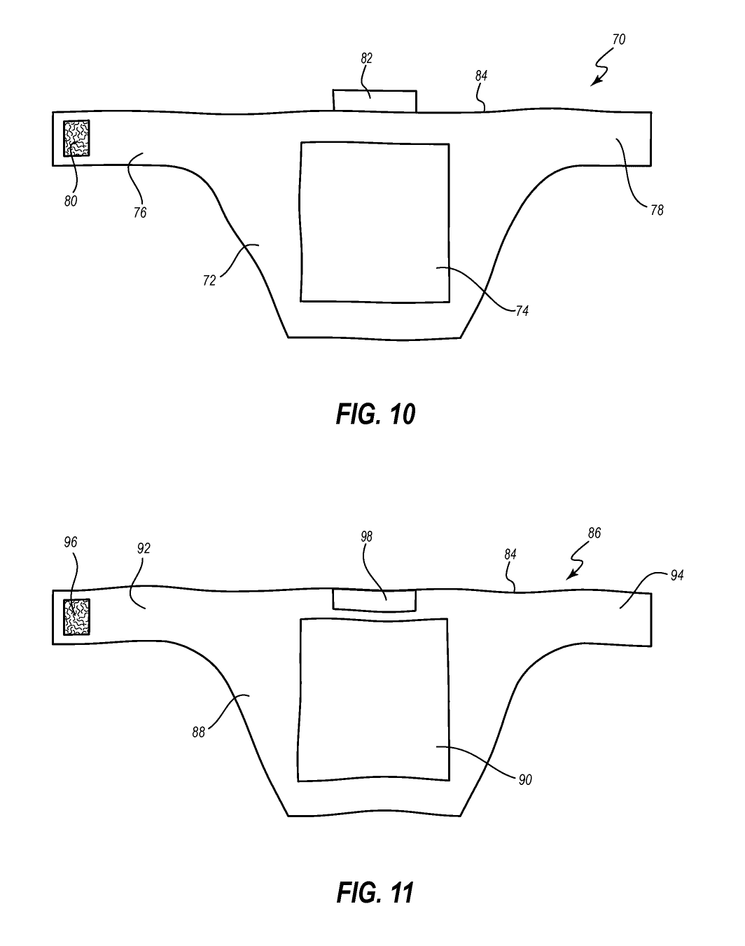

In another embodiment, referring to FIG. 10, a diaper shield 70 may include a cover 72, an absorbent core 74, a first and second strap 76, 78 and a fastener 80 as discussed and described above with respect to the diaper shield 10, above. The diaper shield 70 may include an adhesive tab 82 that may be attached to an inside or outside edge 84 of the cover 72. The adhesive tab 82 is configured to secure the diaper shield 70 to a standard diaper.

In a further embodiment, referring to FIG. 11, a diaper shield 86 may include a liner 88, an absorbent core 90, a first and second strap 92, 94 and a fastener 96 as discussed and described above with respect to the diaper shield 10, above. The diaper shield 86 may include an adhesive tab 98 that may be attached to the liner 88. The adhesive tab 98 is configured to secure the diaper shield 86 to a standard diaper. The adhesive tab 82 or 98 may be made of a material, such as hook and loop fasteners, adhesive tape, or materials known in the art to removably attach two surfaces. The adhesive material may be covered by a release liner that can be removed when ready to attach the diaper shield to a diaper.

In one embodiment, the diaper shield may be in the form of a continuous band. The length of the continuous band generally corresponds to the waist circumference of the wearer. In this aspect, the diaper shield has sufficient stretchability to enable it to be placed around the waist of the wearer by sliding it over the head of the wearer and moving it down the body into location or by sliding it up from the wearer's feet. This aspect may also include an adjustment mechanism to enable the diaper shield to be tightened about the waist of the wearer.

Because the diaper shield is a separate component from the standard diaper with which it may be used, and it does not contact any bodily fluids unless there is leakage from the standard diaper, the diaper shield may be reused. Optionally, the liner and the outer cover of the diaper shield may include materials capable of being laundered so that even a soiled diaper shield can be reused.

The diaper shield may be positioned in overlapping position with the waist edges of the standard diaper worn by a wearer. The width of the straps may be sufficient to cover the waist edges of the standard diaper so as to improve leakage inhibition. The diaper shield may be fastened at any location about the waist, depending on the wearer's desired results. It may be fastened at the front of the wearer so that the center panel will cover the wearer's back to inhibit leakage through the back of the standard diaper. It may also be fastened at the back of the wearer so that the center panel will cover the wearer's front to inhibit leakage through the front of the standard diaper.

The outer cover may be liquid impermeable or permeable. In one embodiment, the outer cover can be made from a thin film, or other flexible, substantially liquid impermeable material. The flexible material may readily conform to the general shape and contour of the wearer's body. The outer cover may prevent various bodily fluids and exudates from wetting or otherwise contaminating various bedding or outer garments worn by the wearer. The outer cover may include a film, such as a polyethylene film. In one embodiment, the outer cover may include a woven or nonwoven fibrous web layer, which is treated or constructed, partially or wholly, to impart the desired levels of liquid impermeability to selected regions that are adjacent to or proximate the absorbent core. The outer cover may include a gas-permeable, nonwoven fabric layer laminated to a polymer film layer which may or may not be gas-permeable.

The outer cover may include an inherently water vapor permeable material or a micro-porous material which permits gases, such as water vapor, to escape from the diaper shield while substantially preventing liquid exudates from passing through the outer cover. The outer cover may be composed of a microporous polymer film or a nonwoven fabric which has been coated or otherwise modified to impart a desired level of liquid impermeability. The outer cover may also be embossed or otherwise provided with a pattern or matte finish to exhibit a more aesthetically pleasing appearance.

The outer cover may be extensible. The outer cover may provide a selected, sustained deformation when subjected to an applied tensile force and then allowed to relax for a selected time period beginning immediately after removal of the tensile force. The extensible outer cover may be composed of a necked fabric, a creped fabric, a micro-pleated fabric, polymer films or the like, as well as combinations thereof.

The liner presents a body-facing surface that is compliant, soft-feeling, and non-irritating to the wearer's skin. The liner may be less hydrophilic than the absorbent core, and is sufficiently porous to be liquid permeable, permitting liquid to readily penetrate through its thickness to reach the absorbent core. The liner may be made from a wide selection of web materials, such as porous foams, reticulated foams, aperture plastic films, natural fibers, synthetic fibers, or a combination of natural and synthetic fibers. The liner may help isolate the wearer's skin from liquid held in the absorbent core. Various woven and nonwoven fabrics can be used for the liner. The liner may be composed of a meltblown or spunbonded web of the desired fibers, and may also be a bonded-carded-web. The liner may be composed of a substantially hydrophobic material, and hydrophobic material may optionally be treated with a surfactant or otherwise process to impart a desired level of wettability and hydrophilicity. The surfactant can be applied by any conventional means, such as spraying, brush coating or the like. The liner can be made of extensible materials.

The absorbent core can be any absorbent material that tends to swell or expand as it absorbs liquid excreted or exuded by the wearer. The absorbent core material may be made of airformed, airlaid and/or wetlaid composites of fibers and high absorbency materials. High absorbency materials typically are made of polyacrylic acids. The fibers may be fluff pulp materials, or any combination of crosslinked pulps, hardwood, softwood, and synthetic fibers. Airlaid and wetlaid structures include binding agents, which are used to stabilize the structure. In addition, various forms, absorbent films, and high absorbency fabrics can be used as an absorbent material.

Various types of wettable, hydrophilic fibrous material can be used to form absorbent core. Examples of suitable fibers include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material which has a suitable hydrophilic moiety and is not readily removable from the fiber, or by sheathing the nonwettable, hydrophobic fiber with a hydrophilic polymer during or after the formation of the fiber. Selected blends of the various types of the fibers mentioned above may also be employed.

What is claimed is:

1. A diaper shield comprising:
an outer cover including a substantially rectangular center panel having a first and a second longitudinal side edges and a first and a second lateral side edges, a first and a second substantially triangular panels connected to the first and the second lateral side edges respectively, a first strap connected to the first triangular panel extending longitudinally outward, a second strap connected to the second triangular panel extending longitudinally outward; and
a liner connected to the outer cover, wherein the liner defines a bodyfacing surface and includes a fluid permeable material.

2. The diaper shield as claimed in claim 1, further comprising an absorbent core disposed between the liner and the outer cover.

3. The diaper shield as claimed in claim 1, further comprising a fastener for connecting the first strap and the second strap.

4. The diaper shield as claimed in claim 1, wherein the liner is in superposed relation to the outer cover.

5. The diaper shield as claimed in claim 1, wherein both the outer cover and the liner include stretchable material.

6. The diaper shield as claimed in claim 1, further comprising an adhesive tab attached to the center panel to adhere the diaper shield to a diaper.

7. The diaper shield as claimed in claim 1, wherein the diaper shield's enclosed length is adjustable.

8. The diaper shield as claimed in claim 1, wherein the outer cover is formed integrally from a single planar member, and the liner is formed integrally from a single planar member.

9. A diaper shield comprising:
an outer cover including a substantially rectangular center panel having a first and a second longitudinal side edges and a first and a second lateral side edges, a first and a second substantially triangular panels connected to the first and the second lateral side edges respectively, a first strap connected to the first triangular panel extending longitudinally outward and with concave shaped peripheral side edge extending between the first strap and the first triangular panel, a second strap connected to the second triangular panel extending longitudinally outward and with concave shaped peripheral side edge extending between the second strap and the second triangular panel; and
a liner connected to the outer cover, wherein the liner includes a fluid permeable material.

10. The diaper shield as claimed in claim 9, further comprising an absorbent core sandwiched between the liner and the outer cover.

11. The diaper shield as claimed in claim 9, further comprising a fastener disposed on the first strap and the second strap to connect the first and second straps together.

12. The diaper shield as claimed in claim 9, wherein at least one of the outer cover and the liner include stretchable material.

13. The diaper shield as claimed in claim 9, further comprising an adhesive tab attached to the diaper shield to removably adhere the diaper shield to a diaper.

14. The diaper shield as claimed in claim 9, wherein the enclosed length of the diaper shield is adjustable.

15. The diaper shield as claimed in claim 9, wherein the outer cover and the liner are made from a single planar member.

16. The diaper shield as claimed in claim 9, wherein the liner is superposed in relation to the outer cover.

17. A diaper shield comprising:
an outer cover including a center panel with opposite a first side edge and a second side edge, a first strap connected to the center panel at the first side edge, an second strap connected to the center panel at the second side edge;
a liner connected to the outer cover, wherein the liner defines a bodyfacing surface and includes a fluid permeable material;
a fastener for connecting the first strap and the second strap; and
means for reducing the center panel's tendency to rotate and move around when worn by a wearer.

18. The diaper shield as claimed in claim 17, wherein both the first and second straps are made of stretchable material.

19. The diaper shield as claimed in claim 17, further comprising an adhesive tab attached to the liner to adhere the diaper shield to a diaper.

20. The diaper shield as claimed in claim 17, wherein the circumference of the diaper shield when worn is adjustable.
